(19) United States
(12) Patent Application Publication
(10) Pub. No.: US 2022/0087815 A1
(43) Pub. Date: Mar. 24, 2022

(54) ARTIFICIAL HEART VALVE AND METHODS FOR IMPLANTING THE SAME

(71) Applicant: VONG MEDICAL LTD., ADI (IL)

(72) Inventors: VADIM BERNSHTEIN, HAIFA (IL); OFER BOGOMOLETZ, ADI (IL)

(21) Appl. No.: 17/423,371

(22) PCT Filed: Mar. 21, 2019
(86) PCT No.: PCT/IL2019/050321

§ 371 Date: Jul. 15, 2021

(51) Int. Cl. A61F 2/24

(52) U.S. Cl. CPC A61F 2/2418; A61F 2230/0065; A61F 2/2436

(57) ABSTRACT

An artificial heart valve to replace a natural valve between an atrium and a ventricle of a heart that comprises a network having a shrunken state and an extended state, wherein the network comprises a plurality of regions differing in flexibility. A plurality of leaflets is attached to the network, and a coat is provided that at least partially is coating the network. A method for implanting the artificial heart valve is described as well.

### FIELD

The present subject matter relates to vascular system implants. More particularly, the present subject matter relates to artificial heart valve implants.

### BACKGROUND

A heart valve, also known as cardiac valve, is a valve controlling one-way flow of blood through the heart. In a mammalian heart there are four types of heart valves: the mitral valve, also known as bicuspid valve, which is between the left atrium and the left ventricle; the tricuspid valve which is between the right atrium and the right ventricle; the aortic valve which is between the left ventricle and the aorta; and the pulmonary valve which is between the right ventricle and the pulmonary artery.

In a patient suffering from valvular heart disease heart valves become dysfunctional, in relation to various physiological conditions. For example, in patients suffering from advanced heart failure, the prevalence of mitral and/or tricuspid regurgitation is high. Furthermore, in patients suffering from mitral regurgitation (MR) blood leaks backwards through the mitral valve when the heart contracts. In some cases, repair of a dysfunctional heart valve solves the problem. However, there are cases in which repair of the dysfunctional heart valve is not enough, and the solution is implantation of an artificial heart valve that replaces the dysfunctional natural heart valve. For example, in some cases, patients suffering from Angina pectoris need to undergo replacement of the heart valve.

The implantation of an artificial heart valve may require open-heart surgery. A preferred alternative is packing the artificial heart valve in a delivery system, inserting the delivery system into a patient's body and transferring the delivery system to the patient's heart through the vascular system, namely through blood vessels, for example veins, that eventually reach the right atrium. The procedure comprises making a cut in the skin at the site of insertion, inserting the delivery system into the vascular system, for example into a vein, transferring the delivery system through the vascular system from the site of insertion to the site of implantation in the heart, ejecting the artificial heart valve from the delivery system and transplanting the artificial heart valve in place.

Implantation of an artificial heart valve by transferring it through the vascular system involves crimping or shrinking of the artificial heart valve in order to facilitate packaging in the delivery system and its transfer through narrow blood vessels. Therefore, implantation of artificial mitral and tricuspid valves is currently a challenging task, primarily due to the large diameter, long length and high rigidity of the crimped or shrunken artificial mitral or tricuspid valve. As a result, the diameter of the delivery system is large as well, thus limiting the transfer of the artificial mitral and tricuspid heart valves through blood vessels to less favorable approaches, like trans-apical and trans-atrial transfer. Due to the aforementioned reasons, the currently available artificial mitral and tricuspid valves are not suitable for more favorable approaches, like trans-femoral, trans-subclavian and trans-jugular vein transfers, therefore eliminating usage of these approaches. Currently available prior art artificial mitral and tricuspid valves are transferred to the site of implantation in the heart by trans-apical and trans-atrial approaches, which are highly risky and involve a high mortality rate. As a result, high risk patients are not assigned to heart valve replacement operation and eventually die due to lack of treatment. Furthermore, the currently available prior art transferring approaches for implantation of artificial heart valves may have deleterious effects. For example, Trans-apical Aortic Valve Replacement (TAVI) with a delivery system having an 18 Fr diameter caused myocardial injury which resulted in apical dysfunction early after the procedure in 28% of the patients. The apical dysfunction was transient in half of the patients and was associated with a decrease in left ventricular function (Israel M. Barbash, Danny Dvir, Itsik Ben-Dor, Paul J. Corso, Steven A. Goldstein, Zuyue Wang, Elizabeth Bond, Petros G. Okubagzi, Lowell F. Satler, Augusto D. Pichard and Ron Waksman, Impact of Trans apical Aortic Valve Replacement on Apical Wall Motion, Journal of the American Society of Echocardiography, Volume 26, Issue 3, March 2013, Pages 255-260).

To summarize, prior art artificial heart valves and their methods of implantation have various disadvantages: they are not suitable for the favorable trans-femoral, trans-jugular and trans-subclavian vein transfer procedures, they have a large diameter in the shrunken state, they are rigid in the shrunken state, they are prone to paravalvular leaks, they may catch natural heart valve chords during implantation, they are prone to functional fracture, they are exposed to fatigue fracture risks, and their usage involve a relatively high mortality rate.

### BRIEF DESCRIPTION OF THE DRAWINGS

Embodiments are herein described, by way of example only, with reference to the accompanying drawings. With specific reference now to the drawings in detail, it is stressed that the particulars shown are by way of example and for purposes of illustrative discussion of the preferred embodiments, and are presented in the cause of providing what is believed to be the most useful and readily understood description of the principles and conceptual aspects of the embodiments. In this regard, no attempt is made to show structural details in more detail than is necessary for a fundamental understanding, the description taken with the drawings making apparent to those skilled in the art how several forms may be embodied in practice.

In the drawings:

FIGS. 1A-B schematically illustrate, according to an exemplary embodiment, a side perspective view and a side view, respectively, of a network of an artificial heart valve in a shrunken state.

FIG. 2 schematically illustrates, according to an exemplary embodiment, a side perspective view of a network of an artificial heart valve in a shrunken state, bent at the second region.

FIG. 3A schematically illustrates, according to an exemplary embodiment, a cross-section view of a network extending forward and FIG. 3B schematically illustrates, according to an exemplary embodiment, a side view of a network configured to extend forward.

FIG. 4A schematically illustrates, according to an exemplary embodiment, a cross-section view of a network extending backward and FIG. 4B schematically illustrates, according to an exemplary embodiment, a side view of a network configured to extend backward.

FIG. 5A schematically illustrates, according to an exemplary embodiment, a cross-section view of a network where some struts extend forward and some struts bend backward and FIG. 5B schematically illustrates, according to an exemplary embodiment, a side view of a network where some struts are configured to extend forward and some struts are configured to extend backward.

FIG. 6 schematically illustrates, according to an exemplary embodiment, a cross-section side view of an artificial heart valve in an extended state, implanted on an annulus of a heart valve.

FIG. 7 schematically illustrates, according to an exemplary embodiment, a side perspective view of an artificial heart valve comprising a plurality of artificial leaflets.

FIG. 8 schematically illustrates, according to an exemplary embodiment, a side perspective view of an artificial heart valve comprising a plurality of artificial leaflets attached to a network.

FIG. 9 schematically illustrates, according to an exemplary embodiment, a side view of a network in a shrunken state and attachment points of artificial leaflets on the network.

FIG. 10 schematically illustrates, according to an exemplary embodiment, an upper view of an artificial heart valve in an extended state, looking from an atrium of a heart in which the artificial heart valve is implanted.

FIG. 11 schematically illustrates, according to an exemplary embodiment, a lower view of an artificial heart valve in an extended state, looking from a ventricle of a heart in which the artificial heart valve is implanted.

FIG. 12 schematically illustrates, according to an exemplary embodiment, a side view of an artificial heart valve in an extended state.

FIG. 13 schematically illustrates, according to an exemplary embodiment, a cross-section side view of a heart in which an artificial heart valve is implanted, showing a network of the artificial heart valve and chords and natural leaflets entrapped within the artificial heart valve.

FIG. 14 schematically illustrates, according to an exemplary embodiment, a cross-section side view of a heart in which an artificial heart valve is implanted, showing a network of the artificial heart valve covered with a cover and chords entrapped within the artificial heart valve, while natural leaflets are held by a surface of the covered network.

FIG. 15 schematically illustrates, according to an exemplary embodiment, a cross-section side view of a heart in which an artificial heart valve is implanted, showing a network of the artificial heart valve and natural leaflets and chords are held by a surface of the network.

FIG. 16A schematically illustrates, according to an exemplary embodiment, a side view of a network and FIG. 16B illustrates a zoom-in image of front edges of struts of a first region of the network.

FIGS. 17A-F schematically illustrates, according to an exemplary embodiment, a side cross-section view of a heart during stages of a method for implanting an artificial heart valve.

### DESCRIPTION OF THE PREFERRED EMBODIMENTS

Before explaining at least one embodiment in detail, it is to be understood that the subject matter is not limited in its application to the details of construction and the arrangement of the components set forth in the following description or illustrated in the drawings. The subject matter is capable of other embodiments or of being practiced or carried out in various ways. Also, it is to be understood that the phraseology and terminology employed herein is for the purpose of description and should not be regarded as limiting. In discussion of the various figures described herein below, like numbers refer to like parts. The drawings are generally not to scale.

For clarity, non-essential elements were omitted from some of the drawings.

The present subject matter provide an artificial heart valve that has the following advantages over prior art artificial heart valves: The artificial heart valve of the present subject matter is suitable for trans-femoral, or trans-subclavian, or trans-jugular vein catheterization procedure; it has a small diameter in the shrunken state; it is at least partially rigid and flexible in a shrunken state as well as in an extended state when it is implanted in place; it is not prone to leakage of blood between in its contact area with the annular tissue; it does not catch natural heart valve chords during implantation; it is not prone to functional fracture due to structural robustness of the artificial heart valve of the present subject matter; its functionality is not expected to be influenced in the case of fracture due to material's fatigue, and its usage may involve a very low mortality rate. Additional advantages of the artificial heart valve of the present subject matter over prior art artificial heart valves are described hereinafter.

According to one embodiment, the present subject matter provides an artificial heart valve configured to be in a shrunken state and an extended state. The artificial heart valve in the shrunken state is configured to be transferred through a patient's body, for example through blood vessels, from an insertion site to an implantation site in the heart. The artificial heart valve in the extended state is configured to be fixed to a heart valve annulus and replace the natural heart valve. According to another embodiment, the artificial heart valve is flexible and elastic in the shrunken state. Therefore, the artificial heart valve in the shrunken state is configured to be transferred through blood vessels while managing to go through the turns and curves that are typical to blood vessels. Thus, in contrast to prior art heart valves, the artificial heart valve of the present subject matter is configured to be transferred through blood vessels, particularly through the trans-femoral vein, the trans-subclavian vein and the sub-jugular vein. According to yet another embodiment, the artificial heart valve, in the shrunken state, may have any diameter. In other words, in contrast to prior art artificial heart valves that have relatively large diameters in a shrunken state that render them not suitable for trans-femoral, trans-subclavian and trans-jugular vein transfer, the artificial heart valve of the present subject matter may have any diameter, inter alia a diameter that is suitable for trans-femoral, trans-subclavian and trans-jugular vein transfer.

According to one embodiment, the artificial heart valve is configured to replace a mitral heart valve, and accordingly such valve may be designated hereinafter “artificial mitral heart valve”. According to another embodiment, the artificial heart valve is configured to replace a tricuspid heart valve, and accordingly such valve may be designated hereinafter “artificial tricuspid heart valve”.

According to one embodiment, the artificial heart valve is configured to withstand blood pressures to which the natural heart valve that is replaced by the artificial heart valve is exposed. For example, the artificial heart valve is configured to withstand a normal blood pressure gradient of substantially 120 mm Hg for example; a moderate-severe hypertension blood pressure of substantially 180 mm Hg for example; and a very severe hypertension blood pressure of substantially 210 mm Hg, for example.

According to another embodiment, the artificial heart valve is elastic and as default is in the extended state. In other words, the artificial heart valve has a shape memory in the extended state. Therefore, there is a need to exert some force in order to bring the artificial heart valve from the extended state to the shrunken state, because once it is allowed to be free the artificial heart valve tends to return to the extended state. There is also a need for exerting some force for keeping the artificial heart valve at the shrunken state. Once this force is released, the artificial heart valve returns to the default extended state due to its elasticity and shape memory in the extended state. According to another embodiment, the artificial heart valve comprises a network. As described hereinafter, the structure of the network and the material of which the network is made are responsible for the elasticity and shape memory of the network and generally of the artificial heart valve.

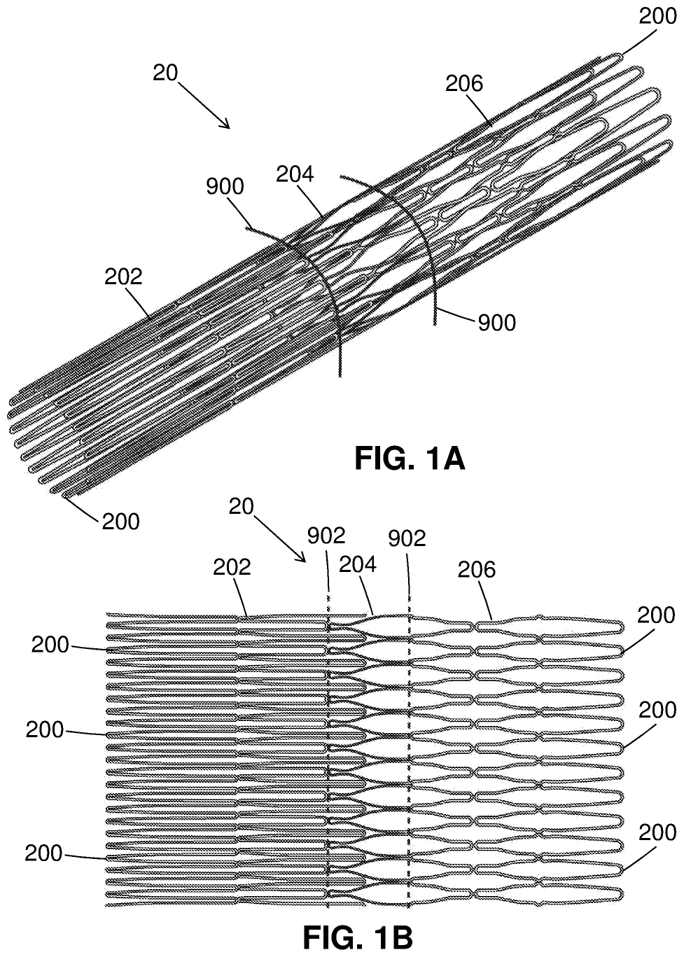

FIGS. 1A-B schematically illustrate, according to an exemplary embodiment, a side perspective view and a side view, respectively, of a network of an artificial heart valve in a shrunken state. According to one embodiment, the network 20 is configured to serve as a skeleton of the artificial heart valve, namely serve as a supporting and anchoring element for the artificial heart valve. According to another embodiment, in the shrunken state, the network 20 has a substantially cylindrical structure. According to yet another embodiment, the network 20 comprises a plurality of regions differing in flexibility. According to still another embodiment, the network comprises a first region 202, a second region 204 and a third region 206. The regions of the network 20 are schematically separated in FIG. 1A with solid lines 900 and in FIG. 1B with broken lines 902. During transfer through the vascular system, the first region 202 is at the front side relative to the direction of transfer and the third region 206 is at the rear side relative to the direction of transfer.

The network 20 is essentially a network of a plurality of struts 200 interconnected in a web-like structure. A strut 200 is a structural element configured to resist pressure in the direction of its length. As illustrated for example in FIGS. 1A-B, there is a difference in the structure and number of struts 200 between the three regions of the network 20. According to one embodiment, the number of struts 200 in the first region 202 is higher than in the third region 206. According to another embodiment, the number of struts 200 in the first region 202 is twice as high as in the third region 206. For example, the first region may comprise 48 struts 200 while the third region 206 may comprise 24 struts 200. According to a further embodiment, the struts 200 in the first region 202 are thicker than the struts 200 in the third region 206.

According to one embodiment, ideally, the struts 200 are straight. However, according to another embodiment, the struts 200 may be bent, dependent on the loads exerted on the struts 200 and the bending of the struts 200. It should be noted, though, that such bending of the struts 200 does not influence the functionality of the struts 200 and the artificial heart valve 1 in general.

According to one embodiment, of the plurality regions of the network 20, an internal region is the most flexible.

According to another embodiment, of the three regions of the network 20, the third region 206 is the most rigid, the second region 204 is the most elastic and flexible and the first region 206 is moderately elastic and flexible.

According to one embodiment, when the artificial heart valve is in the shrunken state, the first region 202 of the network 20 is configured to be at the front side in the direction of transfer of the artificial heart valve within a delivery system to its site of implantation. As such, the first region 202 has a certain level of elasticity and flexibility in order to allow maneuvering of the artificial heart valve within the blood vessels. This is achieved due to a high level of discretization in the first region 202, comprising relatively thick and short struts, compared to the third region 206 that comprises thinner and longer struts, as can be seen for example in FIG. 1B.

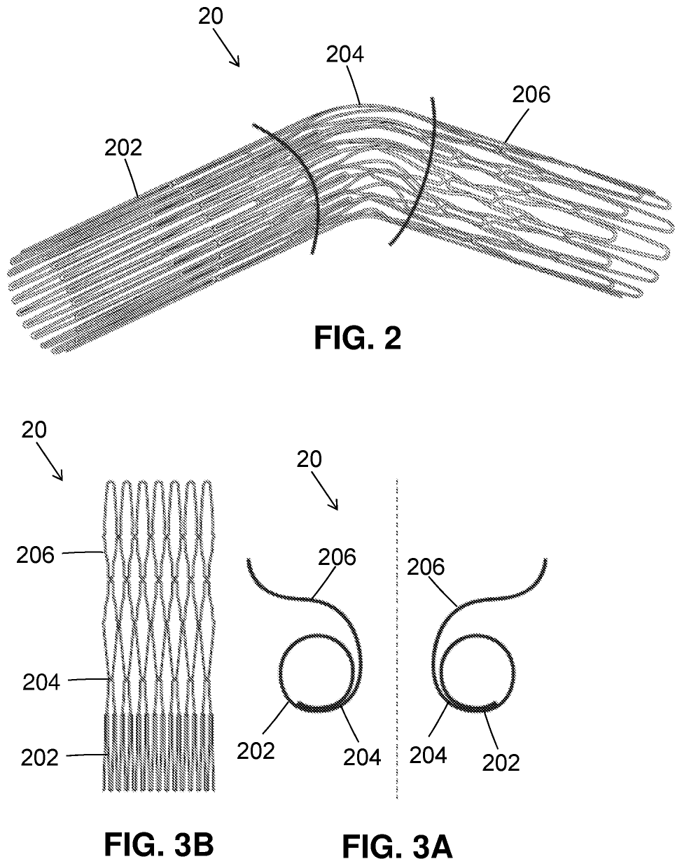

FIG. 2 schematically illustrates, according to an exemplary embodiment, a side perspective view of a network of an artificial heart valve in a shrunken state, bent at the second region. Since the second region 204 of the network 20 is the most elastic and flexible region of the three regions of the network 20, the second region 204 is configured to easily bend while the network 20 is at the shrunken state, as illustrated in FIG. 2. This feature of the second region 204 of the network 20 allows easy transfer of the artificial heart valve within a delivery system in its way to an implantation site in the heart, especially smoothly passing through bent blood vessels.

According to one embodiment, in the shrunken state the second region 204 has a flexibility higher than the flexibility of the first region 202 and the third region 206. According to another embodiment, the second region 204 is configured to facilitate bending of the artificial heart valve in the shrunken state. According to yet another embodiment, due to its flexibility and bending ability, the second region 204 is configured to facilitate navigation of the artificial heart valve in the shrunken state through blood vessels during its transfer to its implantation site.

During implantation of the artificial heart valve, the network 20 extends from a shrunken state to an extended state. The network 20 may extend in various ways, giving rise to different structures of the extended network. The way of extension and the final structure of the extended network 20 are controllable. FIGS. 3-5 schematically illustrate various embodiments of the way of extension of the network 20 and the structure of the struts 200 of the network that determines each way of extension.

In this regard it should be mentioned again that in order for the network 20 to stay in a shrunken state it has to be held in a element that holds it in the shrunken state, for example a delivery system that has a hollow-tube structure. The network 20 in the shrunken state is held inside the delivery system. When the network 20 is ejected through one side of the delivery system, the first region 202 of the network 20 is ejected first, followed by the second region 204 and then by the third region 206. Once the network 20 is ejected from the delivery system, the parts that are outside the delivery system assume their default structure, namely the extended state. As mentioned above, there are various embodiments of the extended structure that the network 20 assumes during the extension, that occurs during the ejection of the network 20 from the delivery system.

FIG. 3A schematically illustrates, according to an exemplary embodiment, a cross-section view of a network extending forward and FIG. 3B schematically illustrates, according to an exemplary embodiment, a side view of a network configured to extend forward.

According to one embodiment, the network 20 extends forward when ejected from a delivery system, as illustrated in FIG. 3A. In other words, all the struts 200 of the network 20 extend forward. As a result, the network 20 extends to a mushroom-like structure. The manner of bending of the second region 204 determines the manner of bending of the network 20, while the manner of bending of the second region 204 is determined by the design of the second region 204. During the forward bending, the second region 204, together with the first region 202 roll around themselves. When the network 20 extends forward, it exerts a radial force. FIG. 3B illustrates the structure of struts 200 of a network 20 that is configured to bend forward during extension. The struts 200 of the second region 204 are relatively longer than struts 200 of a network configured to bend backward, as illustrated in FIG. 3B.

FIG. 4A schematically illustrates, according to an exemplary embodiment, a cross-section view of a network extending backward and FIG. 4B schematically illustrates, according to an exemplary embodiment, a side view of a network configured to extend backward.

According to another embodiment, the network 20 extends backward when ejected from a delivery system, as illustrated in FIG. 4A. In other words, all the struts 200 of the network extend backward. As a result, the network extends to an umbrella-like structure. Also in this case, the manner of bending of the second region 204 determines the manner of bending of the network 20, while the manner of bending of the second region 204 is determined by the design of the second region 204. During the backward bending, the second region 204 makes a U-turn 700, causing the first region 202 bend backward in a direction opposite to the direction of ejection of the network 20 from a delivery system, the direction illustrated with arrow 800. As a result of bending backward, the network 20 exerts an axial force and becomes rigid and resistant to bending.

In the forward bending the network 20 exerts a force in one direction, while in the backward bending the struts exert force in another direction. There is another embodiment, where some of the struts 200 of the network 20 bend forward and some of the struts 200 bend backward.

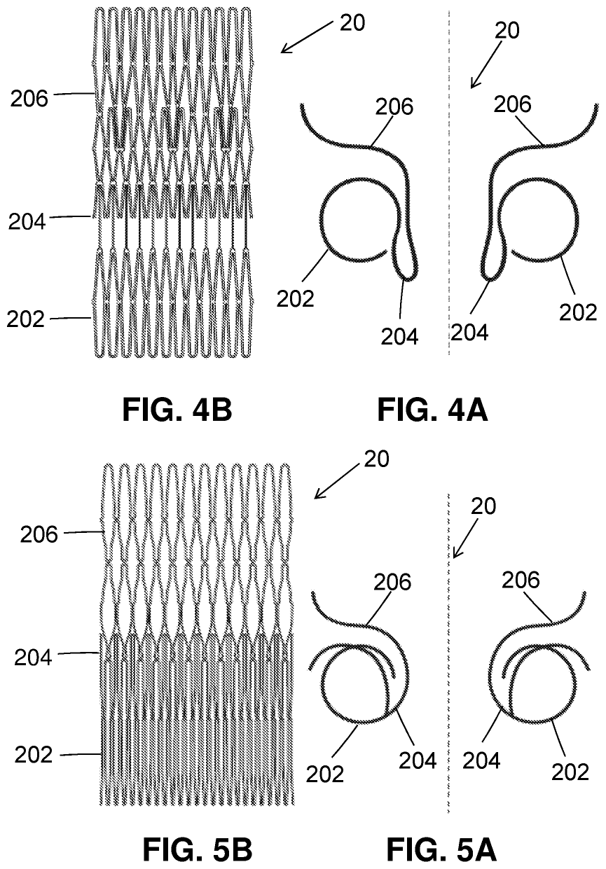

FIG. 5A schematically illustrates, according to an exemplary embodiment, a cross-section view of a network where some struts extend forward and some struts bend backward and FIG. 5B schematically illustrates, according to an exemplary embodiment, a side view of a network where some struts are configured to extend forward and some struts are configured to extend backward.

According to yet another embodiment, some struts 200 of the network 20 are configured to extend and bend forward and some struts 200 are configured to extend and bend backward. In other words, the bending of the struts 200 of the first region 202 is a mixture of forward bending and backward bending. According to a preferred embodiment, the struts 200 alternately bend in either direction. To illustrate this preferred embodiment, if the struts 200 are numbered in sequence, then for example odd numbered struts 200 bend forward and even-numbered struts 200 bend backward. In either way, the bending 200 of the struts forms a toroidal structure, as with a network 20 where all the struts 200 bend either forward or backward.

The mixed bending forward and backward is preferred compared to the only forward bending and only backward bending, because it is a mixture of both. In the forward bending the struts 200 exert force in one direction and in the backward bending the struts 200 exert force in another direction. In the mixed bending, there is a mixture of forces that are exerted by the struts 200. In this way a tighter attachment of the artificial heart valve to the annulus is achieved, as well as better sealing against blood leakage.

The mode of bending of the struts 200 is determined during the manufacture of the network 20. For example, the width of a strut 200 determines whether the strut will bend forward or backward when extending from the shrunken state. Another importance of this embodiment is that the way of bending of the struts 200 is predictable and adjusted during the manufacture of the network 20, namely the way of bending of the struts 200 is controlled. Furthermore, the struts 200 have a memory of the way of their bending. If, according to one embodiment, the network 200 is made of a material that is rigid at a warm temperature and soft and amenable to structural manipulation at a freezing temperature, so if the network 20 is exposed to a freezing temperature and its structure is changed, once it is exposed again to a warm temperature, the network assumes back the structure and the way of bending of the struts 200 that were determined during the manipulation at the freezing temperature, as described above.

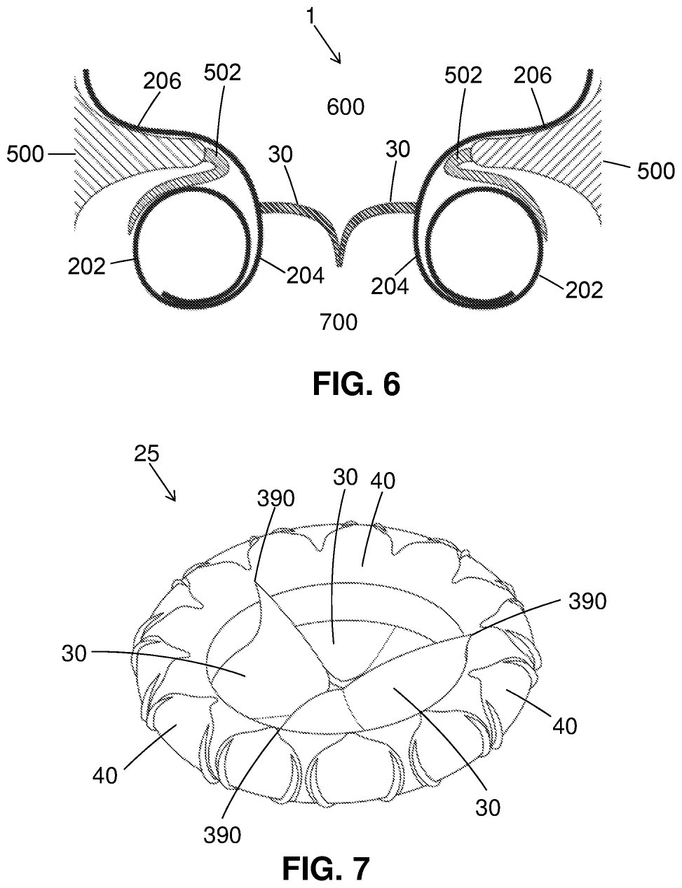

FIG. 6 schematically illustrates, according to an exemplary embodiment, a cross-section side view of an artificial heart valve in an extended state, implanted on an annulus of a heart valve. The annulus 500 illustrated in FIG. 6 may be either a mitral annulus or a tricuspid annulus, since the artificial heart valve 1 may be configured to be implanted on each one of them.

Once the artificial heart valve 1 is ejected from a delivery system, as explained hereinafter, it is anchored over the annulus 500. FIG. 6 illustrates an artificial heart valve 1 that has extended forward, similarly to what is illustrated in FIG. 3A.

During the extension of the artificial heart valve 1 from the shrunken state to the extended state, the second region 204 is configured to facilitate bending of the first region 202 relative to the third region 206 in a manner that the first region 202 is getting closer to the third region 206. As a result, in the extended state, when the artificial heart valve 1 is implanted in place, as can be seen in FIG. 6, it is anchored to the annulus 500, in a manner that the first region 202 attaches to the side of the annulus 500 that faces the ventricle 700 and the third region 206 attaches to the side of the annulus 50 that faces the 600. According to a further embodiment, in the extended state the second region 204 is configured to provide the power needed for holding the first region 202 and the third region 206 one facing the other while anchoring on the annulus 500.

As illustrated in FIG. 6, the first region 202 in the extended state presses against the third region 206 and the annulus 500 is entrapped in-between the first region 202 and the third region 206. This ensures tight sealing and prevention of blood leakage between the artificial heart valve 1 and the annulus tissue 500. In addition, the natural heart valve leaflets 502 are entrapped in-between the bent first region 202. This prevents disturbance to the activity of the artificial heart valve 1 by the natural leaflets 502.

The flexibility of the second region 204 in the shrunken state allows the formation of an artificial heart valve 1 that in its shrunken state is longer than prior art artificial heart valves in the shrunken state. Thus, because of the flexibility of the second region 204 the artificial heart valve 1 can be easily transferred through blood vessels to the implantation site, for example by trans-femoral, trans-jugular and trans-subclavian vein transfer procedures. On the other hand, prior art artificial heart valves that lack this flexibility are either not transferred by trans-femoral, trans-jugular and trans-subclavian vein transfer procedures, or are relatively shorter in the shrunken state, compared to the artificial heart valve 1 of the present subject matter, in order to facilitate their maneuvering through blood vessels. It is obvious that a long artificial heart valve 1 is advantageous over a shorter artificial heart valve when it is installed in place because it has a greater surface area to engage with the tissue adjacent to its implantation site, for example the annulus 500. This in addition allows growth of more tissue that is in contact with the artificial heart valve 1. These advantages are reflected, for example, in better anchoring of the artificial heart valve 1 in place. In addition, during the installation of the artificial heart valve 1 in place, in the shrunken state a long artificial heart valve 1 is advantageous over a shorter artificial heart valve 1, because the ejection and self-folding and placement at the right site are more controllable and predictable with a longer artificial heart valve 1 in the shrunken state compared to shorter ones. In case of a short artificial heart valve —during its ejection from the delivery system it is self-folding and the folding is controllable and predictable until a certain point. However, at a certain point the partially ejected prior art artificial heart valve is folded rapidly without an ability to control its shape and location. This might end up with positioning of the prior art artificial heart valve in a wrong place or in a wrong shape—factors that render the implantation unsuccessful and the function of the artificial heart valve 1, a failure. A short artificial heart valve 1 in the shrunken state may be substantially 40 mm, substantially 45 mm and up to substantially 50 mm long. A long artificial heart valve 1 in the shrunken state may be for example more than substantially 50 mm long, for example substantially 70 mm long.

The flexibility and elasticity properties of the network 20 are determined inter alia by the material of which the network 20 is made. According to one embodiment, the network 20 is made of a material that is configured to be soft and prone to structural manipulation at one range of temperatures, for example at freezing temperature, and rigid and flexible at another range of temperatures, for example ambient temperature and above. More particularly, the material of which the network 20 is made is soft and prone to structural manipulation at substantially 0° C. and lower; and is rigid and flexible at a temperature of a body of an animal in which the artificial heart valve 1 is implanted, for example in the case of a human being—at substantially 37° C. An exemplary material of which the network 20 is made is a shape memory alloy, for example nickel titanium, also known as nitinol.

According to one embodiment, the network 20 in the shrunken state is uni-layered. In other words, in the shrunken state, the artificial heart valve 1 comprises one layer of network 20, as can be seen for example in FIGS. 1A-B. This is advantageous of prior art artificial heart valves that comprise a network having more than one layer in the shrunken state, even in cases when only part of the length of the prior art network 20 has more than one layer. The advantage is expressed in terms of the width of the network 20 in the shrunken state. A network 20 of the present subject matter having only one layer is thinner than a prior art network 20 having more than one layer. Any person skilled in the art would appreciate that a thin network 20 in the shrunken state is advantageous over a wide network 20 in the shrunken state, because it can pass easily through blood vessels.

FIG. 7 schematically illustrates, according to an exemplary embodiment, a side perspective view of an artificial heart valve comprising a plurality of artificial leaflets. As illustrated in FIG. 7, the network 20 (not seen) is in the extended state, forming a frame 25. According to one embodiment, the artificial heart valve 1 comprises a plurality of artificial leaflets 30 attached to the frame, as illustrated for example in FIG. 7. For example, the artificial heart valve 1 illustrated in FIG. 7 comprises three artificial leaflets 30. A border zone between two adjacent artificial leaflets 30 is termed also commissure cooptation zone 390, as illustrated in FIG. 7.

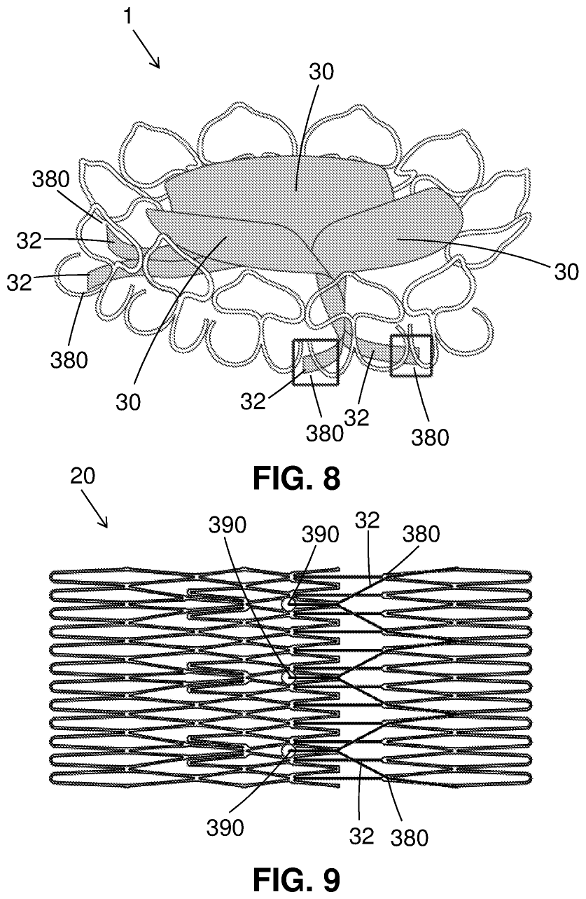

FIG. 8 schematically illustrates, according to an exemplary embodiment, a side perspective view of an artificial heart valve comprising a plurality of artificial leaflets attached to a network. According to one embodiment, an artificial leaflet 30 of the artificial heart valve 1 is attached to the network 20 to at least one attachment point. In the extended state, an artificial leaflet 30 is attached to an internal part of a frame 25 made as a result of bending of the network 20. According to another embodiment, an artificial leaflet 30 is attached to an external part of the frame 25, as can be seen in FIG. 8. Attachment points 380 are present on an external part of the frame 25, and each artificial leaflet 30 is attached to an attachment point 380 with a connecting wire 32. In addition, the artificial leaflets 30 may be attached to the frame 25 in their commissure cooptation zones 390.

FIG. 9 schematically illustrates, according to an exemplary embodiment, a side view of a network in a shrunken state and attachment points of artificial leaflets on the network. According to a preferred embodiment, an artificial leaflet 30 is attached to an internal part of the frame 25 and to an external part of the frame 25. As can be seen in FIG. 9, commissure cooptation zones 390 are present on the network 20 on an area that becomes an internal part of a frame 25 generated by the network 20 in an extended state. In addition, the artificial leaflet 30 is attached, for example with connecting wires 32 to an area of the network 20 that becomes an external part of a frame 25 generated by the framework 20 in an extended state. This is an advantage of the artificial heart valve 1 of the present subject matter over prior art artificial heart valves, in which there is only one point of attachment of an artificial leaflet 30 with the network 20, whereas in the present subject matter there is at least one point of attachment, preferably a plurality of points of attachments, more preferably two points of attachment, with the network 20.

According to one embodiment, the artificial leaflets 30 may be attached to the network 20 at any position or region, for example to the first region 202, or the second region 204 or the third region 206. According to a preferred embodiment, the artificial leaflets 30 are attached to the third region 206.

According to one embodiment, in the extended state of the network 20, as can be seen in FIG. 6, the artificial leaflets 30 are positioned in the appropriate place, and they are configured to allow flow of blood from the atrium 600 to the ventricle 700 and prevent flow of blood from the ventricle 700 to the atrium 600.

According to one embodiment, illustrated for example in FIG. 7, the network 20 of which the frame 25 is made, is covered with a cover 40. The cover 40 is configured to avoid direct touch of the network 20 with heart tissue and avoid flow of blood through the network 20 on the other hand. Furthermore, the cover 40 is configured to bend together with the network 20 while still covering the network 20, whenever the network 20 assumes a new structure, for example when the network 20 bends at the second region 204 in the shrunken state, as illustrated in FIG. 2, or when the network extends from a shrunken state to an extended state. Accordingly, the cover 40 is made of any material that is suitable for any one of the aforementioned embodiments, for example but not limited to, fabric, nylon, treated skin and the like.

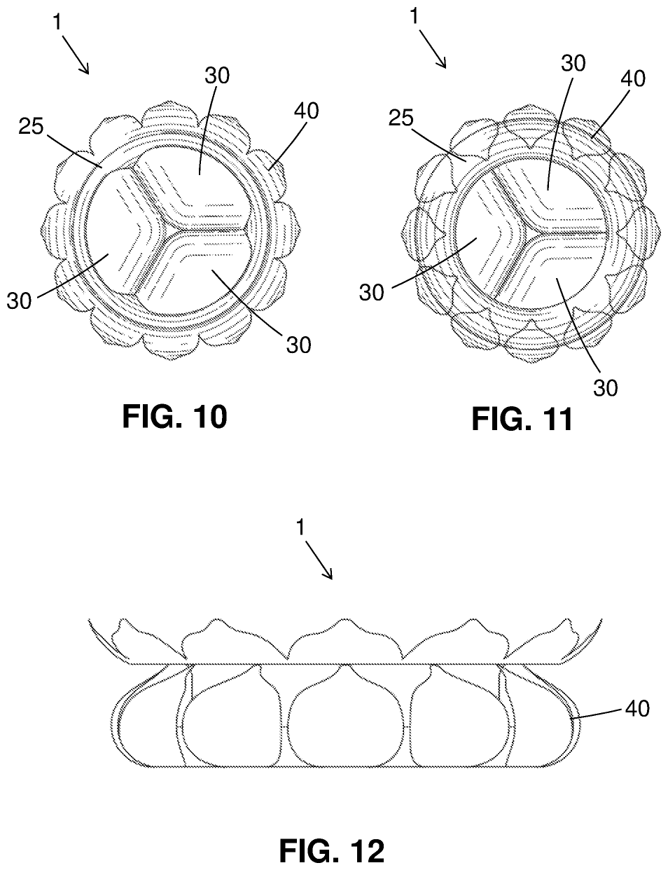

FIG. 10 schematically illustrates, according to an exemplary embodiment, an upper view of an artificial heart valve in an extended state, looking from an atrium of a heart in which the artificial heart valve is implanted.

FIG. 11 schematically illustrates, according to an exemplary embodiment, a lower view of an artificial heart valve in an extended state, looking from a ventricle of a heart in which the artificial heart valve is implanted.

FIG. 12 schematically illustrates, according to an exemplary embodiment, a side view of an artificial heart valve in an extended state.

As can be seen in FIGS. 10 and 11, exemplary three artificial leaflets 30 of the artificial heart valve 1 are seen in a closed state, preventing flow of blood from the atrium to the ventricle, for example during diastole.

According to one embodiment, the network 20 is not entirely covered by the cover 40. Thus, during implantation, as will be described hereinafter, when a partially extended artificial heart valve 1 is pulled upwards from a lower part of the heart ventricle toward the annulus, chords and native leaflets maybe caught in between exposed struts 200 of the network 20. After implantation, the chords that are caught by the artificial heart valve 1 may be torn due to continuous friction with the network 20. But this does not influence the anchoring of the artificial heart valve 1 in place because the artificial heart valve 1 does not depend on the chords for anchoring, in contrast to some prior art artificial heart valves. The artificial heart valve 1 of the present subject matter interacts with the annulus for the purpose of anchoring, as can be seen for example in FIG. 6.

According to one embodiment, the third region 206 serves as a functional region of the artificial heart valve 1 because according to one embodiment the artificial leaflets 30 are attached to the third region 206, and because according to another embodiment the cover 40 is attached to the third region 206. The artificial leaflets 30 that are attached to the third region 206 are the components of the artificial heart valve 1 that exert its functionality. The artificial leaflets 30 are responsible on controlling the direction of the flow of blood through the artificial heart valve 1. The cover 40 that is attached to the third region 206 has a major role in preventing leakage of blood through the artificial heart valve 1 because the cover 40 covers the network 20 and serves as a sealing impermeable agent that covers the network 20 that is permeable to liquid, for example blood. Immediately after the artificial heart valve 1 is ejected from the delivery system and installed in place, as will be described hereinafter, the flow of blood that hits the first region 202 in the ventricle during systole—contraction of the heart, is blocked by the cover 40 and directed towards the open artificial leaflets 30 and towards the atrium. Similarly, during diastole—heart resting, flow of blood that hits the third region 206 in the atrium is blocked by the cover 40 and directed towards the closed artificial leaflets 30 that block the flow of blood towards the ventricle.

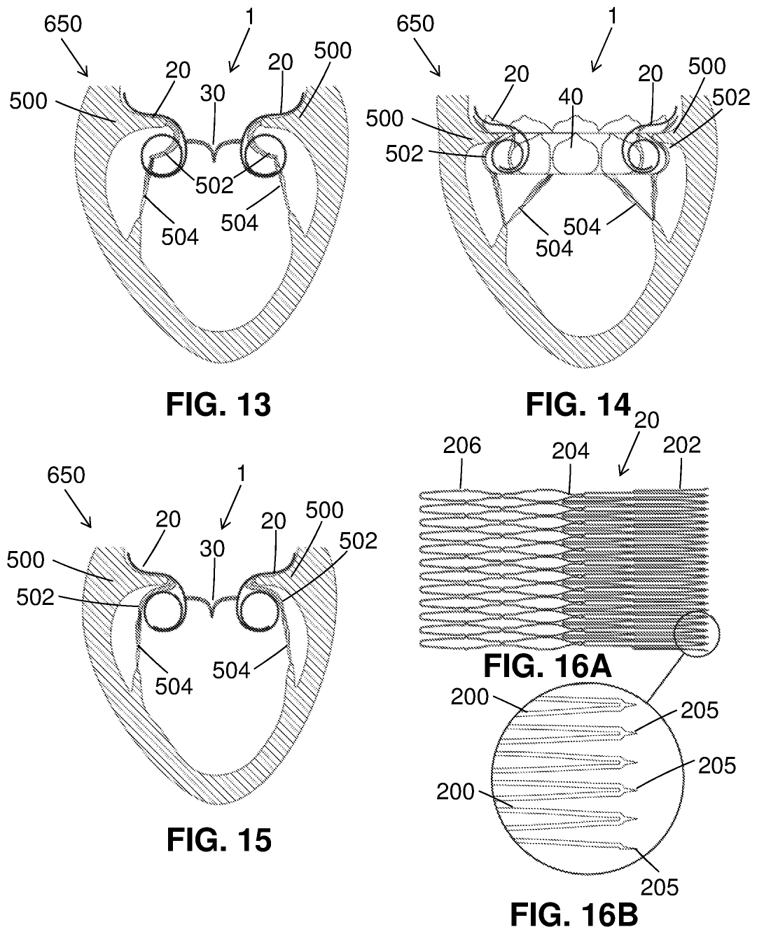

According to one embodiment, the struts 200 of the network 20 are partially covered with the cover 40. In other words, some of the struts 200, as well as the gaps in between the struts, are exposed. Therefore, during the implantation of the artificial heart valve 1 at least some of the chords may be entrapped in the gaps in between the exposed struts 200, and during the extension of the artificial heart valve 1 the at least some of the chords are folded and covered by the bent artificial heart valve 1 in the extended state. FIGS. 13 and 14 illustrate this embodiment.

FIG. 13 schematically illustrates, according to an exemplary embodiment, a cross-section side view of a heart in which an artificial heart valve is implanted, showing a network of the artificial heart valve and chords and natural leaflets entrapped within the artificial heart valve.

This embodiment, of entrapping natural leaflets 502 and chords 504 of the heart 650 in exposed gaps of struts 200 of the network 20 that are not covered by a cover 40, does not allow the chords 504 and natural leaflets 502 to interfere with the normal function of the artificial heart valve 1 of the present subject matter, in contrast to prior art artificial heart valves that do not deal with the problem of free chords that may interfere with the normal function of the artificial heart valve 1. The artificial heart valve 1 of the present subject matter is anchored to the annulus 500, while the natural leaflets 502 and chords 504 are entrapped within the artificial heart valve 1. This is achieved by entrapping the natural leaflets 502 and the chords 504 in the gaps between the struts 200 of the network 20 that are not covered by the cover 40, because the cover 40 partially covers the network 20.

FIG. 14 schematically illustrates, according to an exemplary embodiment, a cross-section side view of a heart in which an artificial heart valve is implanted, showing a network of the artificial heart valve covered with a cover and chords entrapped within the artificial heart valve, while natural leaflets are held by a surface of the covered network. FIG. 14 shows the cover 40 that covers the network 20. According to the embodiment illustrated in FIG. 14, the natural leaflets 502 of the heart 650 are pushed against the annulus 500 by the surface of the cover 40 of the artificial heart valve 1, similarly to the embodiment illustrated in FIG. 6. However, chords 504 are entrapped within the artificial heart valve 1 because the network 20 is partially covered with the cover 40, thus allowing the chords 504 to be entrapped within exposed gaps of struts 200 that are not covered by the cover 40.

Despite the aforementioned embodiment according to which the struts 200 are partially covered and thus exposed, in the extended state, when the artificial heart valve 1 is implanted in its site, the previously exposed struts 200 may no longer be exposed because in the fully folded artificial heart valve 1 the part of the exposed struts 200 may be at least partially covered by another part of the artificial heart 1 that is covered. Therefore, also chords 504 that are entrapped in a gap between exposed struts 200 are covered in the fully extended state, thus eliminating disturbance of the function of the artificial heart valve 1 by the chords 504.

FIG. 15 schematically illustrates, according to an exemplary embodiment, a cross-section side view of a heart in which an artificial heart valve is implanted, showing a network of the artificial heart valve and natural leaflets and chords are held by a surface of the network. In this embodiment, the cover 40 that covers the network 20 is not shown. According to the embodiment illustrated in FIG. 15, the natural leaflets 502 and the chords 504 of the heart 650 are pushed against the annulus 500 by the surface of the artificial heart valve 1, similarly to the embodiment illustrated in FIG. 6. Also in this embodiment, similarly to the previous embodiments, the natural leaflets 502 and the chords 504 do not interfere with the function of the artificial heart valve 1.

The artificial heart valve 1 has a cylinder-like structure in the shrunken state, as illustrated for example in FIG. 1A. Even though FIG. 1A illustrates the network 20 of the artificial heart valve 1, it determines the structure of the artificial heart valve 1, because in the artificial heart valve 1 the network is only coated with a coat 40 and comprises artificial leaflets 30, which essentially do not influence the structure. After its extension, for example during implantation, the artificial heart valve 1 assumes a toroidal structure, as illustrated for example in FIG. 7. A toroid is a surface of revolution with a hole in the middle, like a doughnut, forming a solid body. During extension, the toroidal structure is first formed as a result of bending of the struts 200 in the first region 202 once the first region 202 is ejected from a delivery system. There are several embodiments of bending of the network 20, including bending of the first region 202, that are illustrated in FIGS. 3A, 4A and 5A.

As can be seen for example in FIG. 6, the struts 200 of the second region 204 are flexible in a way that once the third region 206 is ejected from the delivery system the struts 200 of the second region 204 bend in a manner that brings the third region 206 to tightly press against the first region 202. As a result, the artificial heart valve 1 is tightened over the annulus 500. An important outcome of this is that leakage of blood between the artificial heart valve 1 and the annulus 500 is prevented. This is one of the advantages of the artificial heart valve 1 of the present subject matter over prior art artificial heart valves, as some of them do not properly address the issue of leakage after implantation. Therefore, leakage of blood is a major cause of mortality among patients with implanted prior art artificial heart valves.

During the extension of the network 20 from the shrunken state to the extended state, the length of the network 20 is shortened. According to one embodiment, the length of the network 20 is shortened by shortening the struts 200. According to another embodiment, the length of the network 20 is shortened by bending of the network 20 during the extension of the network 20 from the shrunken state to the extended state, several embodiments of which are illustrated in FIGS. 3A, 3B and 3C. The network 20 is bent and assumes a toroidal structure, as illustrated for example in FIG. 7.

Returning now to FIGS. 13-15, according to one embodiment, during implantation and extension of the artificial heart valve 1, the chords 504 and the natural leaflets 502 of the natural heart valve are rejected aside by the partially extended artificial heart valve 1 and eventually the chords 504 and natural leaflets 502 are held between the artificial heart valve 1 and the annulus 500, as illustrated in FIG. 15. According to another embodiment, the chords 504 and natural leaflets 502 are entrapped within the bent artificial heart valve 1, as illustrated in FIG. 13. According to yet another embodiment, the chords 504 are entrapped within the artificial heart valve 1, while the natural leaflets 502 are held between the artificial heart valve 1 and the annulus, as described in FIG. 14. These three embodiments eliminate interference of the function of the artificial heart valve 1 by the chords 504 and natural leaflets 504 and prevent leakage between the artificial heart valve 1 and the annulus 500, because in these three embodiments the chords 504 and natural leaflets 502 are not free to engage with the artificial heart valve 1, particularly with the artificial leaflets 30, but rather held away from the artificial leaflets 30 either between the artificial heart valve 1 and the annulus 500, or within the bent extended artificial heart valve 1.

To summarize, the artificial heart valve 1 may deal with the natural leaflets 502 in any one of the three ways described above and illustrated in FIGS. 13-15. In any case, the result is that in one hand the natural leaflets 502 do not disturb the function of the artificial heart valve 1, and a better sealing against blood leakage is achieved because the entrapped natural leaflets 502 contribute to the sealing, either between the artificial heart valve 1 and the annulus 500 when the natural leaflets 502 are entrapped between the artificial heart valve 1 and the annulus 500, or within the artificial heart valve 1 when the natural leaflets 502 are entrapped within the artificial heart valve 1.

It should be noted that after the implantation of the artificial heart valve 1, the chords 504 and the natural leaflets 502 disintegrate in time. However, as long as they are still intact they may interfere with the artificial heart valve 1, as happening with prior art artificial heart valves. The aforementioned embodiments of the artificial heart valve 1 of the present subject matter show that the present subject matter overcomes this problem and eliminates interference of the functionality of the artificial heart valve 1 by the chords 504 and natural leaflets 502. Thus, the artificial heart valve 1 of the present subject matter is less prone to malfunction and blood leakage than prior art artificial heart valves. As a result, the mortality rate after implantation may decrease dramatically after implantation of an artificial heart valve 1 of the present subject matter, compared to prior art artificial heart valves.

Because of the high strength of the chords 504, some prior art artificial heart valves are designed to attach to the chords 504 and use them as an anchoring support. However, in reality these prior art artificial heart valves do not rely only on the chords 504 for anchoring but also on the natural leaflets 502. The result was that the natural leaflets 502 did not withstand the very high loads exerted on them by the prior art artificial heart valves and eventually the natural leaflets 502 were torn within 2-3 days after implantation, long time before a new tissue has developed over the prior art artificial heart valve. This caused leakage of blood through the prior art artificial heart valve, lung edema and sometimes death.

In contrast to prior art artificial heart valves, the artificial heart valve 1 of the present subject matter does not rely neither on the chords 504, nor on the natural leaflets 502, for anchoring, but rather on the annulus 500, as illustrated for example in FIG. 6. This is an advantage over prior art artificial heart valves that rely on the chords 504 or the chords 504 and the natural leaflets 502 for anchoring. The annulus 500 is much more durable than the natural leaflets 502 and chords 504. In addition, prior art artificial heart valves that do not rely on the annulus 500 for anchoring tend to allow leakage of blood between the prior art artificial heart valve and the annulus 500. In contrast, since the artificial heart valve 1 of the present subject matter relies on the annulus 500 for anchoring, it is tightly attached to the annulus 500—a factor that contributes to the prevention of blood leakage between the artificial heart valve 1 and the annulus 500. Furthermore, in order to eliminate disturbance of the function of the artificial heart valve 1 and in order to prevent blood leakage, during implantation, when the artificial heart valve 1 is ejected from the delivery system and assumes the extended state it catches the chords 504 and natural leaflets 502, or attaches them to the annulus 500, as described above. In either way, not only that the anchoring of the artificial heart valve 1 relies only on the annulus 500 and not on the natural leaflets 5025 and chords 504, but also the natural leaflets 502 and chords 504 do not disturb with the function of the artificial heart valve 1. Furthermore, since the natural leaflets 502 are now either caught within the extended artificial heart valve 1 of attached to the annulus by the artificial heart valve 1, or both, they assist in preventing leakage through the artificial heart valve 1 or between the artificial heart valve 1 and the annulus 500. In this case the natural leaflets 502 serve as a filling material that prevents blood leakage at least until a new tissue develops over the artificial heart valve 1.

During the delivery of the artificial heart valve 1 to its implantation site, as described hereinafter, the delivery system enters an atrium. Then, according to one embodiment, the shrunken artificial heart valve 1 is ejected from the delivery system, passes through the natural leaflets 502 and inserted into the corresponding ventricle. More particularly, since the first region 202 of the network is firstly ejected, front edges of the struts 200 of the first region 202 should pass through the natural leaflets 502 in their way into the corresponding ventricle. According to one embodiment, the front edges of at least some of the struts 200 of the first region 202 are configured to puncture the natural leaflets 502 in order to facilitate the passage of the ejected artificial heart valve 1 through the natural heart valve. Any structural feature known in the art of the front edges of the struts 200 of the first region 202 that confers the front edges of the struts 200 the ability to puncture the natural leaflets 502 is under the scope of the present subject matter, for example sharp tips of any kind as illustrated in FIG. 16B. Puncturing of the natural leaflets 502 not only facilitates passage of the artificial heart valve 1 through the natural heart valve during implantation, but also encourages growth of tissue in the area of the natural heart valve adjacent to the artificial heart valve 1.

FIG. 16A schematically illustrates, according to an exemplary embodiment, a side view of a network and FIG. 16B illustrates a zoom-in image of front edges of struts of a first region of the network. As can be seen in FIG. 16B, the front edges of the struts 200 of the first region 202 comprise sharp tips 205 that are configured to puncture natural leaflets 502. It should be emphasized that sharp tips 205 at the front edges of the struts 200 of the first region 202 are only an exemplary embodiment, and that any means known in the art that is configured to facilitate passage of the artificial heart valve 1 through natural leaflets 502 is under the scope of the present subject matter.

According to one embodiment, sharp tips 205 at the front edges of struts 200 of the first region 20 may have a role in catching the natural leaflets 502 and holding them when the artificial heart valve 1 bends. Thus, when the artificial heart valve 1 bends and rolls, for example as illustrated in FIGS. 3A. 4A and 5A, the caught natural leaflets 502 are entrapped within the extended artificial heart valve 1, as described for example in FIGS. 14-16.

According to another aspect, the present subject matter provides a method for implanting an artificial heart valve 1, the method comprising:

inserting a guide tube into an atrium of a heart;

positioning a front exit of the guide tube close to a natural heart valve of the atrium;

ejecting a delivery system, in which there is an artificial heart valve in a shrunken state, through the front exit of the guide tube;

bringing a front exit of the delivery system to a position between chords and an apex of the heart;

ejecting the artificial heart valve in the shrunken state through the front exit of the delivery system, while allowing the artificial heart valve to extend from the shrunken state to an extending state;

pulling the delivery system backward toward an annulus;

completely ejecting the artificial heart valve through the front exit of the delivery system while allowing the artificial heart valve to completely extend from the shrunken state to the extended state, and further allowing the artificial heart valve in the extended state to anchor to the annulus;

pulling the delivery system back inside the guide tube, and

pulling the guide tube out of the heart.

According to one embodiment, after positioning a front exit of the guide tube close to a natural heart valve, the method for implanting an artificial heart valve comprises: setting the guide tube in a manner that avoids movement of the guide tube.

According to another embodiment, after positioning a front exit of the guide tube close to a natural heart valve, the method for implanting an artificial heart valve comprises: inserting the guide tube into a ventricle under the atrium through the natural heart valve in between the atrium and the ventricle.

According to one embodiment, the artificial heart valve 1 is configured to be transferred to the implantation site through the femoral vein. According to another embodiment, the artificial heart valve 1 is configured to be transferred to the implantation site through the angular vein. According to yet another embodiment, the artificial heart valve 1 is configured to be transferred to the implantation site through the jugular vein. These embodiments are possible due to the flexibility of the second region 202 that facilitates navigation of the artificial heart valve 1 in the shrunken state through blood vessels toward the implantation site.

In any case, if the artificial heart valve 1 is transferred through veins it enters firstly into the right atrium of the heart. When the artificial heart valve 1 is an artificial tricuspid valve 1 it is transferred directly to the natural tricuspid valve that separates between the right atrium and the right ventricle. When the artificial heart valve 1 is an artificial mitral valve 1 it is inserted into the left atrium from the right atrium through the septum that separates between the right atrium and the left atrium, for example by puncturing the septum. Then the artificial mitral valve 1 is transferred to the natural mitral valve that separates between the left atrium and the left ventricle.

According to one embodiment, a guide tube is first inserted through veins into the right atrium. In prior art methods for transferring objects through blood vessels, for example implanting an artificial heart valve, the guide tube is configured to protect blood vessels from puncturing with a delivery system or a catheter that is delivered inside the guide tube, especially when the delivery system or the catheter encounter a turn in a blood vessel. There is a possibility that an operator performing the method would not notice the turn in the blood vessel and puncture the blood vessel. For such cases a guide tube is used. The front edge of the guide tube is not sharp and the guide tube is flexible, thus allowing maneuvering of the guide tube within the blood vessels without hurting them. When the guide tube is brought to place, the delivery system or catheter is pushed inside the guide tube towards its desired site. In this way, the blood vessels are protected against possible damage by the delivery system or the catheter.

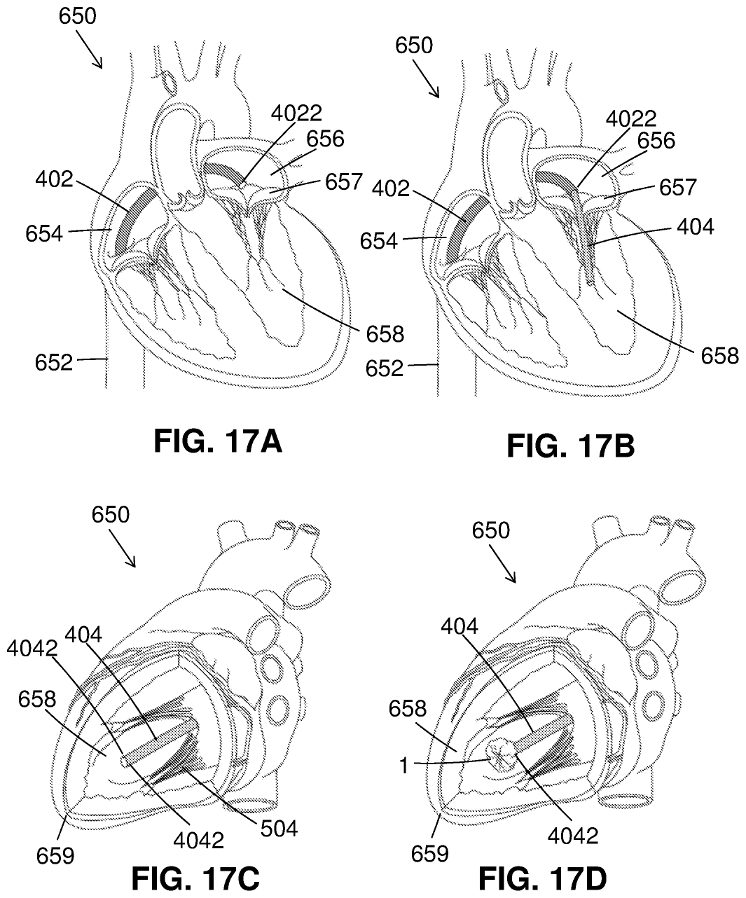

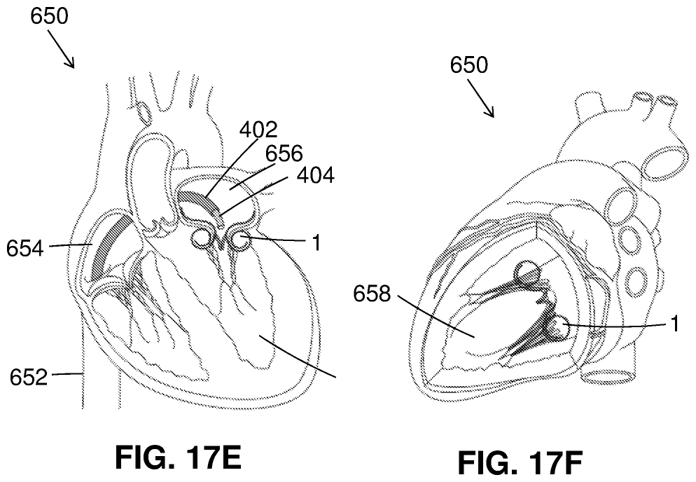

FIGS. 17A-F schematically illustrates, according to an exemplary embodiment, a side cross-section view of a heart during stages of a method for implanting an artificial heart valve. It should be noted that the embodiments illustrated in FIGS. 17A-F of the method for implanting an artificial heart valve 1 are only exemplary, because they are specific to the implantation of an artificial mitral valve 1. Other embodiments of the method for implanting an artificial heart valve 1 are also under the scope of the present subject matter, as described herein.

In FIG. 17A a guide tube 402 is inserted through a vein, for example the inferior vena cava, into the right atrium 654. Then the septum between the right atrium 654 and the left atrium 656 is punctured and the guide tube is inserted into the left atrium 656 through the punctured septum. As can be seen in FIG. 17A, a front exit 4022 is brought close to the natural mitral valve 657.

In FIG. 17B, a delivery system 404 is ejected from the guide tube 402 and inserted into the left ventricle 658 through the natural mitral valve.

In FIG. 17C, the delivery system 404 is further inserted in the left ventricle 658 towards the apex 659.

In FIG. 17D the artificial heart valve 1, in this case the artificial mitral valve 1, is ejected from the delivery system 404 through a front exit 4042.

In FIG. 17E the artificial heart valve 1, in this case the artificial mitral valve 1, is implanted in place after it was completely ejected from the delivery system 404, and the delivery system 404 is returned back to the guide tube 402.

In FIG. 17F the guide tube 402 is completely pulled out of the heart 650 and the artificial heart valve 1, in this case the artificial mitral valve 1, is implanted in place, on the annulus of the natural mitral valve.

The aforementioned stages, illustrated in FIGS. 17A-F, which are briefly described, are described in more detail hereinafter.

A difference between the method of implanting an artificial heart valve 1 of the present subject matter and a prior art method for implanting a prior art artificial heart valve is that in the present subject matter the guide tube 402 is brought very close to the implantation site in the heart 650, namely just above the natural heart valve, where the delivery system 404 is ejected from the guide tube 402 and moved just a little into the ventricle. This is because according to one embodiment, the guide tube 402 of the present subject matter is flexible, thus allowing steering and maneuvering of the guide tube 402 through blood vessels until it reaches a position very close to the implantation site. On the other hand, in prior art implantation methods the guide tube is brought to a blood vessel outside the heart, and there the delivery system is ejected from the guide tube and inserted into the heart towards the implantation site, namely the natural heart valve. This is because prior art guide tubes are rigid and not flexible enough to allow them to be steered and maneuvered through blood vessels until they reach a position very close to the implantation site. From this difference stems the functional difference between the guide tube 402 of the present subject matter and the guide tube of prior art systems and methods for implanting an artificial heart valve. The function of the prior art guide tube is to prevent damaging of blood vessels by the delivery system during the maneuvering of the delivery system in blood vessels toward the implantation site, as outlined above. On the other hand, the function of the guide tube 402 of the present subject matter is indeed to guide the delivery system 404 to the implantation site, as outlined above.

During implantation, the artificial heart valve 1 is ejected from the delivery system 404 in a position lower than the chords 504, towards the apex 659, in order to prevent contact between the artificial heart valve 1 and the chords during its ejection from the delivery system 404. According to one embodiment, the artificial heart valve 1 is ejected from the delivery system 404 adjacent to the apex 659. According to another embodiment, the artificial heart valve 1 is ejected from the delivery system 404 beyond two thirds of the depth of a ventricle. After the first region 202 is ejected and bent, the delivery system 404 and the artificial heart valve 1 partially ejected from it are pulled backward towards the annulus. According to one embodiment, during the pulling backward towards the annulus, the ejection of the artificial heart valve 1 continues. According to another embodiment, during the pulling backward towards the annulus, the artificial heart valve 1 does not continue to be ejected. During the pulling upwards, the artificial heart valve 1 continues to fold around itself while entrapping the natural leaflets 502 and chords 504 as described above. This is an advantage over prior art artificial heart valves with which there is a possibility that during implantation a chord may be caught by the artificial heart valve, causing the artificial heart valve to be locked in place without being able to be removed. This of course may end with a fatal fate to the patient during operation. Therefore, the artificial heart valve 1 of the present subject matter may dramatically reduce the mortality rate of patients during implantation of an artificial heart valve 1.

According to one embodiment, it is possible to trace the position of the guide tube 402 during implantation of the artificial heart valve 1, by echo, also known as ultrasound. This ability assists the operator during the implantation, for example assisting in positioning of the guide tube 402 in the atrium, just above the natural heart valve. This ability also assists navigating the guide tube 402 through blood vessels from the site of entrance into the body until the site in the heart where the delivery system 404 is ejected from the guide tube 402.

As described above, according to one embodiment, the guide tube 402 is inserted into an atrium and then the delivery system 404 is ejected from the guide tube 402 and inserted into the corresponding ventricle through the natural heart valve. According to another embodiment, the guide tube 402 is inserted into an atrium and then further into the corresponding ventricle through the natural heart valve. Also in this embodiment, the guide tube 404 is inserted until it hits the apex 659 and returned slightly backward. Only then the delivery system 404 is ejected from the guide tube 402 and the artificial heart valve 1 is ejected from the guide tube 402.

In prior art methods for implanting an artificial heart valve, imaging devices and methods, like echo, also known as ultrasound, are used for tracing the position of the guide tube, the delivery system and the artificial heart valve during implantation. Imaging devices and methods are also used during the artificial heart valve implantation method of the present subject matter. However, there is a difference in the role of the imaging devices and methods between the prior art and the present subject matter. In the prior art methods for artificial heart valve implantation, the usage of imaging devices and methods is mandatory, because an operator that operates the implantation needs imaging in order to see the position of the guide tube, delivery system and artificial heart valve during implantation in order to decide whether to perform the next stop or not. Imaging is the only way to monitor the progress of the implantation, in prior art artificial heart valve implantation methods. On the other hand, in the method of the present subject matter, imaging is optional. There is no mandatory need for imaging for performing the artificial heart valve implantation. The reason for this is that the operator physically feels the position of the components during implantation. This is based on the difference between the prior art implantation method and the method of the present subject matter. In the method of the present subject matter, according to one embodiment, the guide tube 402 is inserted into an atrium and then a delivery system 404 is ejected from the guide tube 402 and inserted from an atrium into a corresponding ventricle and further until it reaches the apex 659. Then, the delivery system 404 is pulled slightly backwards. An operator can easily fill when the delivery system 404 touches the apex 659, because he simply cannot push the delivery system 404 further. In addition, a slight push of the delivery system 404 against the apex 659 does not injure the apex because of the thickness of the apex 659 and the flexibility and relative softness of the delivery system. In addition, during delivery through blood vessels and in the atrium, the flexibility and relative softness of the guide tube 402 allow easy navigation of the guide tube 402 through the winding blood vessels without worrying about injuring the blood vessels. Thus, usage of imaging during the implantation method of the present subject matter is optional and may be used only for verifying the sensation of the operator. On the other hand, in prior art methods there is a need to closely monitor the position of the guide tube, delivery system and artificial heart valve during implantation, otherwise blood vessels may be injured and the artificial heart valve may not be positioned in the right place. For example, in a prior art method where the apex 659 is punctured and the delivery system or the guide tube are inserted through the punctured apex 659 into a ventricle and then towards the annulus or even further into a corresponding atrium, the position of the edge of the guide tube, for example, covered with a tip, should be continuously monitored by imaging. If an operator of this prior art method would rely only on his sensation, as in the method of the present subject matter, he may puncture the annulus or the atrium thin wall, thus putting the patient in danger. Such incidents are avoided in the present subject matter.

According to one embodiment, the front exit 4022 of the guide tube 402 is inserted into a ventricle until the front exit 4022 touches the apex 659. Then it is slightly pulled backwards before the delivery system 404 is ejected though the front exit 4022 of the guide tube 402 and the artificial heart valve 1 is ejected from the delivery system 402.

According to one embodiment, the guide tube 402 is inserted into the atrium and placed adjacent to the annulus. According to a preferred embodiment, the guide tube 402 is placed at the center of the atrium. According to yet another embodiment, the guide tube 402 is inserted into the ventricle through the annulus. According to a preferred embodiment, the guide tube 402 is inserted into the ventricle through the center of the annulus. According to a further embodiment, the guide tube 402 is inserted into the ventricle until the front exit 4022 of the guide tube 402 touches the apex 659 and then the guide tube 402 is puled slightly back toward the annulus, in a manner that places the front exit 4022 of the guide tube 402 between the chords 504 and the apex 659.

When the front exit 402 of the guide tube 402 is positioned in place, as may be verified, for example, by using echo, also known as ultrasound, the guide tube 402 is fixed in place in a manner that prevents movement of the guide tube 402. Any method and means known in the art for fixing the guide tube 402 in place is under the scope of the present subject matter, for example but not limited to, fixing the part of the guide 402 tube that is outside the body of the patient undergoing the implantation to the skin of the patient with a plaster for example, using a locking mechanism for fixing the guide tube 402—for example a gripping element, and the like. The guide tube 402 is fixed in place in order to prevent un-controlled movement of the guide tube 402 during the ejection of the delivery system 404 and subsequently the ejection of the artificial heart valve 1. If the guide tube 402 is not fixed in place, it may move and as a result the artificial heart valve 1 may be ejected and implanted in a non-desired place, or even cause damage, for example by catching chords 504 un-intentionally or puncturing a wall of the heart 659.

According to one embodiment, after the guide tube 402 is fixed, the delivery system 404 is ejected from the guide tube 402. No matter what is the position of the open exit 4022 of the guide tube 402—an atrium, an annulus or a ventricle, after the delivery system 404 is ejected from the guide tube 402 it is inserted to a position where the front exit 4042 of the delivery system 404 is placed between the chords 504 and the apex 659. The reasoning behind this embodiment is that it is desired that once the artificial heart valve 1 is ejected from the delivery system 404 and opened, the artificial heart valve 1 will not interact with the chords 504. Any manner for positioning the front exit 4042 of the delivery system 404 between the chords 504 and the apex 659 is under the scope of the present subject matter, for example—inserting the delivery system 404 until it touches the apex 659 and then pulling the delivery system 404 slightly back towards the annulus 500, monitoring the position of the delivery system 404 while inserted into the ventricle until it is determined that the front exit 4042 of the delivery system 404 is positioned between the chords 504 and the apex 659, and the like.

Once the delivery system 404 is positioned in the desired place, the artificial heart valve 1 is ejected from the delivery system 404.

When the artificial heart valve 1 is transferred to its implantation site it is ejected from the delivery system 404. Since the network 20 is coated with a coat 40 there are not any opened and sharp ends of struts 200 of the network 20 exposed. Therefore, the chance for catching chords 504 by the artificial heart valve 1 is minimized, and even entirely prevented. This renders the implantation process of the artificial heart valve of the present subject matter safer and more reproducible compared to prior art artificial heart valve implantation process. As a result, in patients in which an artificial heart valve 1 of the present subject matter is implanted—the mortality rate is expected to dramatically decrease compared to patients in which a prior art artificial heart valve is implanted.

After the guide tube 402 is fixed, the delivery system 404 is ejected from the guide tube 402 through the front exit 4022. In prior art delivery systems, configured to deliver an artificial heart valve, there is a tip attached to the front exit 4042 of the delivery system 404. Since the prior art guide tube 402 is inserted only until a position in a blood vessel, for example a vein, upstream to the heart, and the delivery system is ejected there from the guide tube and inserted into an atrium, the front exit of the delivery system may puncture the blood vessel, for example a vein, through which the delivery system is transferred, or the thin wall of the atrium and cause a serious damage to the patient. Therefore, it is a common practice to cover the front exit of prior art delivery system with a flexible tip, in order to prevent damage to the blood vessel through which the delivery system is transferred or to the thin wall of the atrium. However, a person having knowledge in the art would appreciate the argument that adding the tip to the delivery system 404 renders the system and its operation cumbersome, since the tip adds to the length of the delivery system 404. In contrast, there is no need to cover the front exit 4042 of the delivery system 404 of the present subject matter with a tip, since according to one embodiment, the guide tube 402 is inserted into the heart 650 until its front exit 4022 is positioned adjacent to the natural heart valve, for example in the ventricle close to the apex 659. Thus, the delivery system 404 is ejected from the guide tube 402 directly into the ventricle, where the walls of the ventricle are thick and relatively resistant to damage, not the mention that the guide tube 402 is made of a flexible material configured not to cause damage to blood vessels and the heart.

According to one embodiment, during the ejection and folding of the artificial heart valve 1, specifically during folding of the first region 202, the delivery system 404 together with the artificial heart valve 1 that is still attached to the delivery system 404, are pooled towards the annulus and the atrium. Thus, according to this embodiment, the ejection of the artificial heart valve 1 and the pulling of the delivery system 404 toward the annulus and atrium occur simultaneously. According to another embodiment, the ejection of the artificial heart valve 1 and the pulling of the delivery system 404 toward the annulus and atrium occur separately. For example, at least part of the first region 202 is ejected from the delivery system 404. Then the ejection of the artificial heart valve 1 from the delivery system 404 is halted, and then the delivery system 404 together with the artificial heart valve 1 that is still attached to it is pulled toward the annulus and atrium.

During the ejection of the artificial heart valve 1 from the delivery system 404 the artificial heart valve 1 folds. According to one embodiment, the folding of artificial heart valve 1, particularly the folding of the first region 202, occurs between the chords 504 and the apex 659. This avoids interaction between the chords 504 and the folding artificial heart valve 1. After the artificial heart valve 1 is partially ejected from the delivery system 404, the delivery system is pulled toward the annulus and the atrium. At this time the chords 504 are entrapped by the folded first region 202 in a manner that avoids disturbance of the function of the artificial heart valve 1 by the chords 504. The natural leaflets 502 may also be entrapped by the folded first region 202.

After the artificial heart valve 1 is partially ejected from the delivery system 404 it is entirely ejected from the delivery system 404. At this stage the artificial heart valve 1 is anchored in place by itself due to its self-folding. This is advantageous over prior art artificial heart valves, which do not anchor in place by themselves. Prior art artificial heart valves require three-dimensionally bringing the artificial heart valve exactly to the place of implantation, not to mention the high level of professionalism of the operator that implants the artificial heart valve that is required.

An advantage of the artificial heart valve 1 of the present subject matter over prior art artificial heart valves is that during implantation the implantation is reversible, whereas the implantation of prior art artificial heart valves is not reversible. The reason for this advantage is the type of interaction between the artificial heart valve 1 and the chords 504 and/or the natural leaflets 502. During the implantation of the artificial heart valve 1 of the present subject matter, the chords 504 and/or natural leaflets 502 are entrapped by the folding artificial heart valve 1 during its ejection from the delivery system 404. The chords 504 and/or natural leaflets 502 are entrapped in a reversible manner. Thus, if it is decided for some reason to discontinue the implantation the artificial heart valve 1 is simply pulled back into the delivery system. During the pulling back the artificial heart valve 1 unfolds and transforms to the shrunken state inside the delivery system 404, and the chords 504 and/or the natural leaflets 502 are easily released from the artificial heart valve 1 and eventually separate from it. In addition, when the artificial heart valve 1 of the present subject matter is ejected from the delivery system 404 between the chords 504 and the apex 659, it does not engage with chords 504. Therefore, if it is desired to discontinue the implantation, the artificial heart valve 1 of the present subject matter may easily be returned into the delivery system 404 without any concern about chords 504 and/or natural leaflets 502 that may interrupt because this occurs in an area of the ventricle where there are no chords 504 and/or natural leaflets 502. This allows safe and easy removal of the delivery system 404 together with the artificial heart valve 1 inside it, without any concern. In contrast, implantation of prior art artificial heart valves is not reversible because once the prior art artificial heart valve is ejected from the delivery system it interacts with the chords 504 and/or the natural leaflets 502, either deliberately because it uses the chords 504, for example, for anchoring, or accidently. This interaction does not allow returning of the prior art artificial heart valve into the delivery system in cases when it is needed.

One of the embodiments that allows returning of the artificial heart valve 1 of the present subject matter when it is partially ejected from the delivery system 404 and entrapping chords 504 and/or leaflets 502 is that the struts 200 of the network do not comprises open cells, only closes cells. As can be seen for example in FIG. 1B, the struts 200 form spaces or holes or gaps that are entirely enclosed by a strut 200. Therefore, there are no free ends of struts 200 that may engage with chords 504 and/or natural leaflets 502 and or any tissue in the heart like the annulus 500, in a manner that does not allow release of the network 20 from the engaged chords 504 and/or natural leaflets 502, or the heart tissue. The only place where there are free ends of struts 200, even comprising sharp tips 205, is the front edge of the first region 202, as illustrated in FIG. 16. When the first region 202 is ejected from the delivery system 404, according to one embodiment, in the atrium above the annulus, the sharp tips 205 of the struts 200 at the edge of the first region 202 are configured to puncture the natural leaflets 502 in order to allow penetration of the delivery system 404 into the ventricle through the natural leaflets 502. Once the first region 202 is further ejected from the delivery system 404 it folds in a manner in which the free sharp tips 504 are covered by the folded artificial heart valve 1. On the other hand, there are prior art artificial heart valves that are configured to engage, for example with the annulus, for the purpose of strengthening their anchoring. This is achieved by forming sharp tips, or hook-like elements, or barbs, to a network of the artificial heart valve. These sharp tips, or hook-like elements, or barbs engage with the heart tissue, for example the annulus, and contribute to the anchoring of the prior art artificial heart valve. It is obvious to a person skilled in the art that once these sharp tips, or hook-like elements, or barbs are exposed when the prior art artificial heart valve is ejected from the delivery system, that it would be impossible to reverse the ejection and return the prior art artificial heart valve into the delivery system, because the sharp tips, or hook-like elements, or barbs engage with a heart tissue and get anchored in place.

Within 2-7 days after implantation of the artificial heart valve 1 the chords 504 may be torn. Afterwards, new chords 504 grow over the artificial heart valve 1. These renewed chords 504 assist in the anchoring of the implanted artificial heart valve 1 in place, as well as in preventing leakage of blood.

It is appreciated that certain features of the subject matter, which are, for clarity, described in the context of separate embodiments, may also be provided in combination in a single embodiment. Conversely, various features of the subject matter, which are, for brevity, described in the context of a single embodiment, may also be provided separately or in any suitable sub combination.

Although the subject matter has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. An artificial heart valve configured to replace a natural valve between an atrium and a ventricle of a heart, the artificial heart valve comprising:
a network configured to be in a shrunken state and an extended state, and wherein the network comprises a plurality of regions differing in flexibility;
a plurality of leaflets attached to the network; and
a coat at least partially coating the network.

2. The artificial heart valve of claim 1, wherein the network comprises a plurality of struts.

3. The artificial heart valve of claim 1, wherein the network comprises a first region, a second region, and a third region.

4. The artificial heart valve of claim 1, wherein the network in the shrunken state is uni-layered.

5. The artificial heart valve of claim 1, wherein the network is configured to be in a shrunken state during transfer of the artificial heart valve through blood vessels to a heart.

6. The artificial heart valve of claim 1, wherein the extended state is a default state of the network.

7. The artificial heart valve of claim 1, wherein the artificial heart valve in the shrunken state is configured to be transferred to a heart through veins.

8. The artificial heart valve of claim 1, wherein the artificial heart valve in the extended state is configured to be implanted in a heart between an atrium and a corresponding ventricle.

9. The artificial heart valve of claim 1, wherein the artificial heart valve in the extended state is configured to be anchored to an annulus.

10. The artificial heart valve of claim 1, wherein the artificial heart valve in the extended state is configured to entrap chords of the natural valve in a manner that prevents interference to the function of the artificial heart valve by the chords.

11. The artificial heart valve of claim 1, wherein the artificial heart valve in the extended state is configured to entrap natural leaflets of the natural valve in a manner the prevents interference to the function of the artificial heart valve by the natural leaflets.

12. The artificial heart valve of claim 1, wherein in the shrunken state of the network, the most flexible region is an internal region.

13. The artificial heart valve of claim 3, wherein in the shrunken state of the network, the second region of the network is more flexible than the first region and the third region of the network.

14. The artificial heart valve of claim 2, wherein in the extended state the struts bend forward and exert a radial force.

15. The artificial heart valve of claim 2, wherein in the extended state the struts bend backward and exert an axial force.

16. The artificial heart valve of claim 2, wherein in the extended state some of the struts bend forward and exert a radial force and some of the struts bend backward and exert an axial force.

17. The artificial heart valve of claim 1, wherein in the extended state the network has a toroidal structure.

18. The artificial heart valve of claim 1, wherein the artificial heart valve in the shrunken state is at least substantially 50 mm long.

19. A method for implanting an artificial heart valve, the method comprising:
inserting a guide tube into an atrium of a heart;
positioning a front exit of the guide tube close to a natural heart valve of the atrium;
ejecting a delivery system, in which there is an artificial heart valve in a shrunken state, through the front exit of the guide tube;
bringing a front exit of the delivery system to a position between chords and an apex in a ventricle of the heart;
ejecting the artificial heart valve in the shrunken state through the front exit of the delivery system, while allowing the artificial heart valve to extend from the shrunken state to an extending state;
pulling the delivery system backward toward an annulus;
completely ejecting the artificial heart valve through the front exit of the delivery system while allowing the artificial heart valve to completely extend from the shrunken state to the extended state, and further allowing the artificial heart valve in the extended state to anchor to the annulus;
pulling the delivery system back inside the guide tube; and
pulling the guide tube out of the heart.

20. The method of claim 19, wherein after positioning a front exit of the guide tube close to a natural heart valve of the atrium, the method further comprises:
setting the guide tube in a manner that avoids movement of the guide tube.

21-28. (canceled)
